(19) United States
(12) Patent Application Publication
(10) Pub. No.: US 2024/0226977 A1
(43) Pub. Date: Jul. 11, 2024

(54) FERMENTATION TREATMENT EQUIPMENT AND FERMENTATION TREATMENT METHOD FOR VARIOUS ANIMAL CARCASSES

(71) Applicant: YUNG-LIN HUANG, Huizhou City (CN)

(72) Inventor: YUNG-LIN HUANG, Huizhou City (CN)

(21) Appl. No.: 18/617,507

(22) Filed: Mar. 26, 2024

Related U.S. Application Data
(63) Continuation of application No. 16/941,564, filed on Jul. 29, 2020.

(51) Int. Cl. B09B 5/00; A61L 9/16; A61L 11/00; B09B 3/40

(52) U.S. Cl. CPC B09B 5/00; A61L 9/16; A61L 11/00; B09B 3/40

(57) ABSTRACT

A fermentation treatment equipment for various animal carcasses, including a central controller, a centralized processing warehouse, a plurality of fermentation tanks set in the centralized processing warehouse, and a waste gas treatment device connected with the plurality of fermentation tanks. The fermentation tank includes a tank body and a rotation driving mechanism which drives the tank body to rotate and is electrically connected to a central controller. The tank body is provided with a temperature sensor electrically connected to the central controller, and the rear end of the tank body is provided with an exhaust port connected to the waste gas treatment device. A fermentation treatment method for animal carcasses based on the above equipment. The present disclosure integrates several fermentation tanks into one, simultaneously ferments multiple animal carcasses separately to obtain separate organic fertilizer, which provides convenience for people to handle animal carcasses, reduces cost.

### CROSS REFERENCE TO RELATED DISCLOSURE

This application is a continuation of U.S. application Ser. No. 16/941,564, entitled “FERMENTATION TREATMENT EQUIPMENT AND FERMENTATION TREATMENT METHOD FOR VARIOUS ANIMAL CARCASSES,” filed Jul. 29, 2020, and the entire contents of which are incorporated herein by reference.

### TECHNICAL FIELD

The present disclosure relates to the fermentation field of various animal carcasses, in particular to a fermentation treatment equipment and a fermentation treatment method for various animal carcasses.

### BACKGROUND

The dead bodies of animals often carry a variety of pathogens. If not handled or handled improperly, the corpses will decompose quickly and reek, which will pollute the air, water and soil by pathogenic microorganisms, affect human living environment, and even cause the spread and propagation of diseases.

The most traditional methods of handling animal carcasses are direct landfill or cremation. There will be some invariable problems after the animal carcasses are buried, such as decomposition, putrefaction and reeking, which will lead to the pollution of air, water and soil. However, cremation directly results in the consumption of fuel and a lot of carbon emissions, which is not conducive to environmental protection.

Therefore, it has appeared in the market that high-temperature biodegradation method is used to treat animal carcasses, and finally to prepare organic fertilizer. However, this method is very energy-consuming, contrary to the concept of environmental protection, and can not achieve centralized control.

### SUMMARY

In view of the above deficiencies, the present disclosure is to provide a fermentation treatment equipment and a fermentation treatment method for various animal carcasses, which integrates several fermentation tanks into one, and can simultaneously ferment multiple dead bodies separately to obtain separate organic fertilizer, which provides convenience for people to deal with animal carcasses, and reduces cost. Meanwhile, it is conducive to centralized control to the several fermentation tanks, so as to complete decomposition of animal carcasses with high efficiency, to achieve the object of energy conservation, carbon reduction, and achieve energy regeneration.

The technical solution adopted by the present disclosure to achieve the above object is as follows:

A fermentation treatment equipment for various animal carcasses, including a central controller, a centralized processing warehouse, a plurality of fermentation tanks set in the centralized processing warehouse, and at least one waste gas treatment device connected to the plurality of fermentation tanks, wherein the fermentation tank comprises a tank body base, a tank body arranged on the tank base, and a rotation driving mechanism which drives the tank body to rotate and is electrically connected to the central controller; an inner wall of the tank body is distributed with a plurality of heating components and a plurality of air supply components controlled by the central controller; a temperature sensor electrically connected to the central controller is provided in the tank body, and a rear end of the tank body is provided with an exhaust port connected to the waste gas treatment device.

As a further improvement of the present disclosure, the heating component includes a heating structure distributed on the inner wall of the tank body along a length direction of the tank body, and at least one heating element distributed in the heating structure along a length direction of the heating structure, wherein the heating element is connected to the the central controller via a power supply wire to realize power supply, or the heating element is powered by a microwave wireless power transmission device communicating with the central controller; the air supply component comprises an air pipe arranged on the inner wall of the tank body and distributed side by side with the heating structure, and a plurality of air holes are formed on side wall of the air pipe; a front end of the tank body is provided with a ventilation pipe connected between the air pipe and an external air supply device, and the ventilation pipe is provided with an electric control valve electrically connected to the central controller.

As a further improvement of the present disclosure, a plurality of strip grooves for embedding the heating component and the air supply component are formed on the inner wall of the tank body, and an insulating layer is provided on the inner wall of the tank body.

As a further improvement of the present disclosure, the heating component includes a printed copper foil attached to the inner wall of the tank body, the printed copper foil is connected to the central controller via a power supply wire to realize power supply, or the printed copper foil is powered by a microwave wireless power transmission device communicating with the central controller.

As a further improvement of the present disclosure, a plurality of conical breaking bumps are protruded arranged on the inner wall of the tank body.

As a further improvement of the present disclosure, the rotation driving mechanism includes a motor arranged on the tank body base and electrically connected to the central controller, a transmission shaft connected to an output shaft of the motor, and at least one transmission roller sleeved on a periphery of the transmission shaft and in contact with the outer wall of the tank body.

As a further improvement of the present disclosure, an exhaust structure is arranged at one end of the tank body near the exhaust port, the exhaust structure includes a baffle plate arranged in the tank body and a division plate arranged in the tank body between the baffle plate and the exhaust port, and an exhaust chamber is formed between the division plate and the exhaust port, an internal exhaust port is arranged on the baffle plate, an upper exhaust through hole and a lower exhaust through hole are formed between the division plate and the inner wall of the tank body; an upper exhaust channel and a lower exhaust channel are separately formed between the baffle plate and the division plate, wherein the upper exhaust channel is communicated between the internal exhaust port and the upper exhaust through hole, and the lower exhaust channel is communicated between the internal exhaust port and the lower exhaust through hole; and the internal exhaust port is respectively provided with a upper movable baffle swinging in the upper exhaust channel and a lower movable baffle swinging in the lower exhaust channel.

As a further improvement of the present disclosure, the tank body is mainly composed of a lower tank body and a upper cover covered on a lower tank body; a upper end of the lower tank body is provided with an open input port, and the upper cover is covered on the open input port; and the upper cover is provided with a plurality of hooks.

As a further improvement of the present disclosure, the waste gas treatment device includes an waste gas treatment tank, a plurality of waste gas ventilation pipes connected between a front end of the waste gas treatment tank and the plurality of fermentation tanks, and a negative pressure exhaust fan arranged at a rear end of the waste gas treatment tank, wherein an active bacteria bed is arranged at a front side of an interior of the waste gas treatment tank, a back side of the interior of the waste gas treatment tank is filled with activated carbon; an electric heating component is provided inside the waste gas ventilation pipe; the centralized treatment warehouse is provided with a solar photovoltaic panel which supplies power for the heating component, the electric heating component and the negative pressure exhaust fan, respectively.

A fermentation treatment method for animal carcasses according the above equipment, comprising:

(1) putting a single animal carcass into a numbered fermentation tank, adding the fermentation bacteria and filler, and covering up the fermentation tank;
(2) placing the fermentation tank on a shelf to a corresponding position of a centralized treatment warehouse;
(3) connecting a waste gas ventilation pipe of a waste gas treatment device to an exhaust port of the fermentation tank, and connecting an air pipe of the fermentation tank to an external air supply device;
(4) introducing air into into a tank body through the air pipe under a control of a central controller, and controlling internal heating components to heat a internal temperature of the tank body to 60° C.-70° C.; wherein the central controller controls a rotation driving mechanism to drive the tank body to rotate at a slow speed for 10-30 days; during a rotation of the tank body, the fermentation bacteria in the tank body completely decompose the animal carcass to convert into biological energy, so as to form organic fertilizer and waste gas, and the waste gas is discharged from the exhaust port to the waste gas treatment device;
(5) purifying waste gas by the waste gas treatment device, and finally discharging the purified waste gas into an external environment.

As a further improvement of the present disclosure, the step (4) further includes the following steps: detecting, by a temperature sensor, the temperature inside the tank body in real time, and transmitting a detection result to the central controller in real time; disconnecting power supply for the heating component by the central controller when the temperature inside the tank body is detected to exceed 70° C., and the heating component stops working; supplying power to the heating component by the central controller when the temperature inside the tank body is detected to lower than 60° C., and the heating component start to work; repeating this step continuously to keep the temperature inside the tank body at 60° C.-70° C.

The advantageous effects of the present disclosure are as follows:

(1) Through the design of a centralized processing warehouse that can accommodate a plurality of fermentation tanks, the three-dimensional integration of the plurality of fermentation tanks can reduce the area occupied by fermentation tanks. At the same time, multiple animal carcasses can be fermented separately to obtain separate organic fertilizer, so that people can have equipment to deal with animal carcasses when people encounter animal carcasses, which provides great convenience for people to deal with all kinds of animal carcasses. Moreover, it is advantageous to centralized control and management of the plurality of fermentation tanks;
(2) The special structure design of a plurality of heating components and a plurality of air supply components in the fermentation tank can provide appropriate temperature and oxygen for the fermentation process in the tank, which is conducive to the decomposition and putrefaction of animal bodies inside the tank more completely and thoroughly, and finally to form organic fertilizer, which not only achieves the object of energy saving and carbon reduction, but also realizes energy regeneration;
(3) The waste gas produced by fermentation of the plurality of fermentation tanks is treated by the waste gas treatment device, and the toxic components and odors in the waste gas are absorbed by the active bacteria bed to complete once of the waste gas treatment process; then, the residual pollutants in the waste gas are absorbed by the activated carbon to separate the pollutants from the gas to obtain colorless, odorless and clean gas. And finally, the treated gas is discharged into the environment, and the waste gas treatment is complete and does no harm to the environment and human body;
(4) Through the real-time control of the temperature in the fermentation process, the internal temperature of the tank can be kept at 60° C. to 70° C., which is conducive to the decomposition of animal carcasses by fermentation bacteria inside the tank;
(5) By controlling the rotation speed of the fermentation tank to rotate 2 to 3 turns a day, it not only consumes less electricity, but also saves energy and environmental protection. Due to the rotation of the fermentation tank, the animal carcass, fermentation bacteria and stuffing inside the fermentation tank can be constantly turned, so as to realize the internal oxygen supplement, which accelerates the whole aerobic fermentation process and improves the fermentation efficiency, thereby improving the turnover rate of each fermentation tank.
(6) The current energy consumption of the whole equipment is low, and solar power supply mode can be used to achieve the object of energy conservation and environmental protection.

The above is an overview of the technical solution of the present disclosure. The present disclosure is further described in combination with the attached drawings and specific embodiments.

### BRIEF DESCRIPTION OF THE DRAWINGS

FIG. 1 is a schematic diagram showing the overall structure of the present disclosure;

FIG. 2 is a schematic diagram showing the external structure of the fermentation tank in the present disclosure;

FIG. 3 is a schematic diagram showing the internal structure of the fermentation tank in the present disclosure;

FIG. 4 is a sectional view of a heating component and a gas supply component in the present disclosure;

FIG. 5 is a sectional view of the fermentation tank in the present disclosure;

FIG. 6 is an enlarged view of part A in FIG. 5;

FIG. 7 is the structural diagram of the conical breaking bumps in the tank body of the present disclosure;

FIG. 8 is a structural diagram of the exhaust structure in the present disclosure;

FIG. 9 is the structural diagram showing the upper cover of the tank body in the open state of the present disclosure.

### DETAILED DESCRIPTION OF THE EMBODIMENTS

In order to further illustrate the technical means and efficacy of the present disclosure to achieve the predetermined purpose, the specific implementation mode of the present disclosure is described in detail in combination with the attached drawings and preferred embodiments.

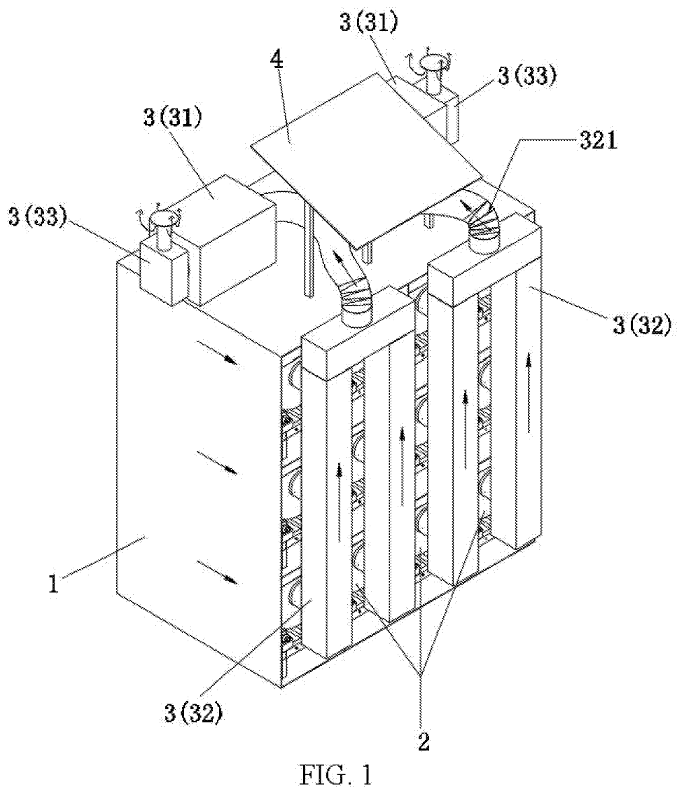

Referring to FIGS. 1 to 3, the present embodiment of the present disclosure provides a fermentation treatment device for various animal carcasses, including a central controller, a centralized processing warehouse 1, a plurality of fermentation tanks 2 arranged in the central processing warehouse 1, and at least one waste gas treatment device 3 connected to the plurality of fermentation tanks 2. Wherein, the fermentation tank 2 includes a tank base 21, a tank body 22 arranged on the tank base 21, and a rotation driving mechanism 23 which drives the tank body 22 to rotate and is electrically connected to the central controller. An inner wall of the tank body 22 is distributed with a plurality of heating components 24 and a plurality of air supply components controlled 25 by the central controller. The plurality of heating components 24 is used to heat the fermentation process in the tank body 22, and the plurality of air supply components controlled 25 provides oxygen for the fermentation process in the tank body 22, which is conducive to the decomposition of the animal carcasses inside the tank body more completely. At the same time, a temperature sensor electrically connected to the central controller is provided in the tank body 22, which is convenient for real-time detection to get the temperature in the tank body 22 and convenient for the control of the internal heating temperature. and a rear end of the tank body 22 is provided with an exhaust port 221 connected to the waste gas treatment device 3. After the fermentation is completed, the waste gas is discharged from the exhaust port 221, and finally the waste gas is carried out the purification treatment by the waste gas treatment device, and finally discharged to the external environment.

In the present embodiment, there are several positions for storing the fermentation tank 2 in the centralized processing warehouse 1. After the fermentation tank 2 is put into the position, the plurality of fermentation tanks 2 can be integrated into one, and multiple animal carcasses can be fermented separately at the same time to obtain separate organic fertilizer, so that people can have equipment to process animal carcasses when they encounter animal carcasses, providing a great convenience for people to handle animal carcasses, and reduces the cost. At the same time, the central controller can automatically and centrally control the work of the plurality of fermentation tanks 2, specifically to control the rotation speed and the internal heating temperature of the fermentation tank 2, and the air (oxygen) introduced into the tank body 22.

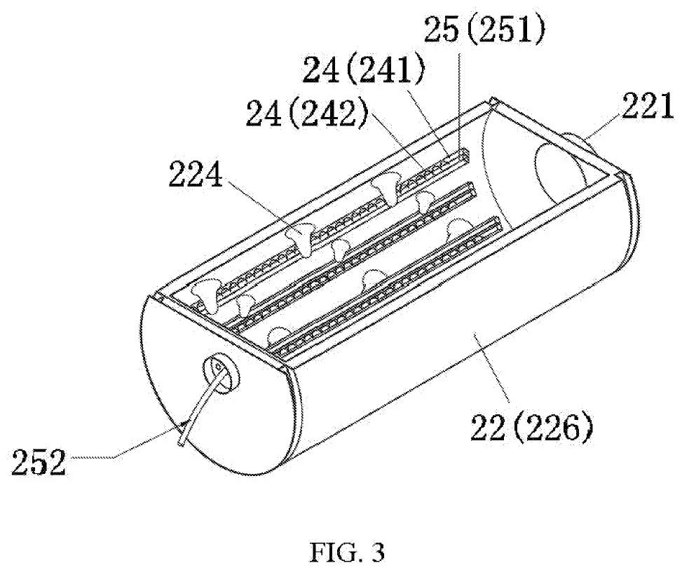

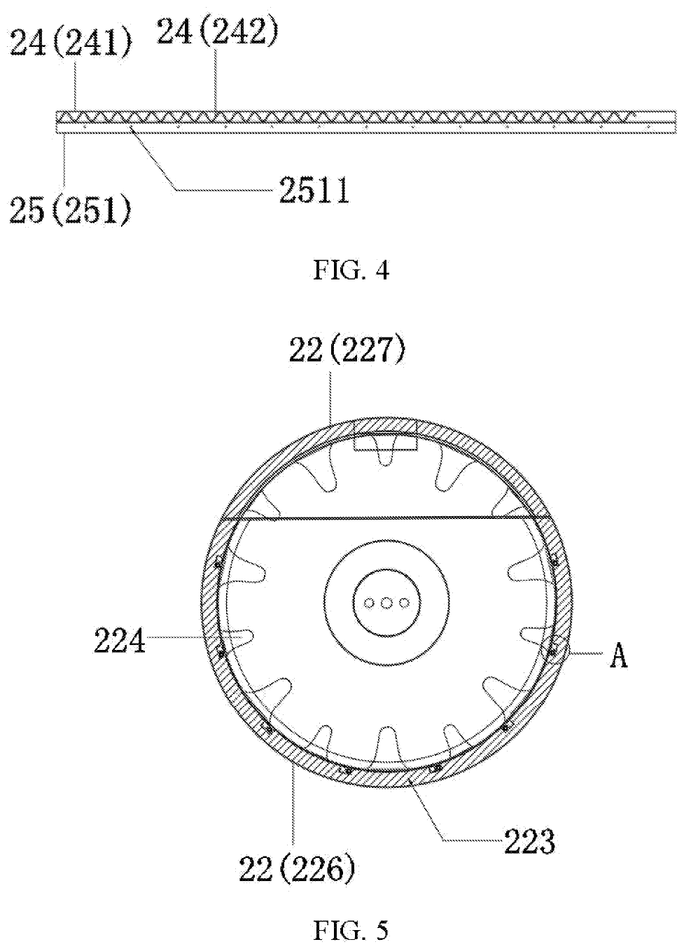

Specifically, as shown in FIG. 3 and FIG. 4, the heating component 24 includes a heating structure 241 distributed on the inner wall of the tank body 22 along the length direction of the tank body 22, and at least one heating element 242 distributed in the heating structure 241 along the length direction of the heating structure 241. Wherein, about the power supply of the heating element 242, it may be provided by wire power transmission mode or microwave wireless power transmission mode, no matter what power supply mode is selected, as long as the heating element 242 can be heated and the temperature in the tank body 22 can be increased. Specifically, the heating structure 241 can be a heating tube, and the heating element 242 can be a heating wire. Specifically, the wire power transmission mode is that the heating element 242 is connected to the the central controller via a power supply wire to realize power supply, while the microwave wireless power transmission mode is that the heating element 242 is powered by a microwave wireless power transmission device communicating with the central controller.

For the structure of the heating component 24, it further includes a printed copper foil attached to the inner wall of the tank body 22, the printed copper foil is connected to the central controller via a power supply wire to realize power supply, or the printed copper foil is powered by a microwave wireless power transmission device communicating with the central controller. The printed copper foil is electrified and heated to evenly heat the inside of the tank body 22, so as to increase the temperature in the tank body 22.

At the same time, the air supply component includes an air pipe 251 arranged on the inner wall of the tank body 22 and distributed side by side with the heating structure 241, and a plurality of air holes 2511 are formed on the side wall of the air pipe 251, as shown in FIG. 4. At the front end of the tank body 22, there is a coupling device for the electrical structure and for oxygen to enter the tank body 22. The front end of the tank body 22 is provided with a ventilation pipe 252 connected between the air pipe 251 and an external air supply device, and the ventilation pipe 252 is provided with an electric control valve electrically connected to the central controller. The ventilation pipe 252 extends out through the coupling device. Air (oxygen) is introduced into the air pipe 251 by the external air supply device through the ventilation pipe 252, and the air is sprayed into the tank body 22 from the plurality of air holes 2511 on the side wall of the air pipe 251, so as to achieve preheating and accelerating the biodegradation reaction, which is conducive to the decomposition of animal carcasses by the fermentation bacteria inside the tank body.

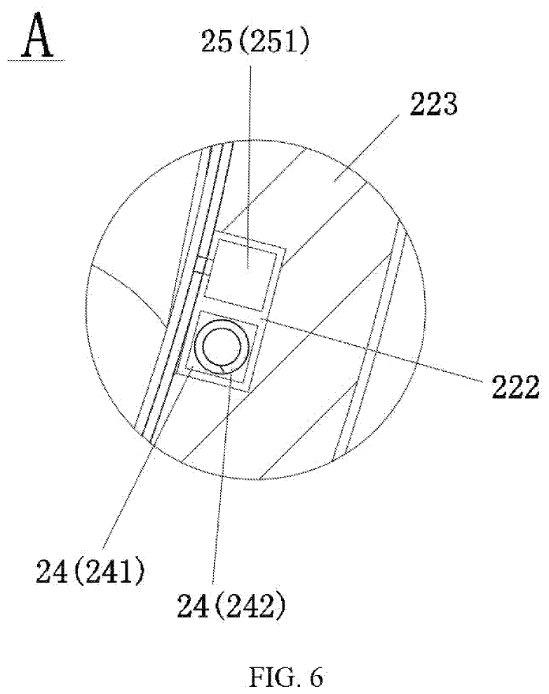

When the heating component 24 and the air supply assembly 25 are installed, a plurality of strip grooves 222 for embedding the heating component 24 and the air supply component 25 are formed on the inner wall of the tank body 22, as shown in FIG. 5 and FIG. 6. At the same time, an insulating layer 223 is provided on the inner wall of the tank body 22, which is beneficial to prevent the internal heat of the tank body 22 from being lost.

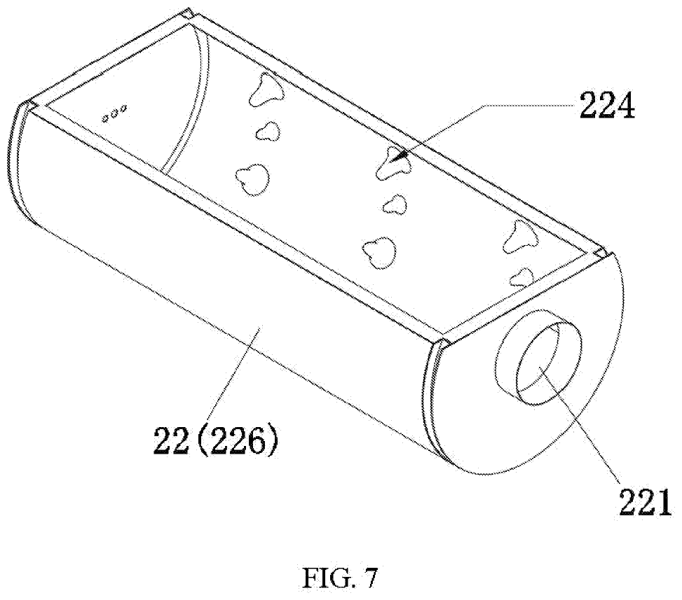

In order to accelerate the decomposition process of animal carcass, a plurality of conical breaking bumps 224 are protruded arranged on the inner wall of the tank body 22 in the present embodiment, as shown in FIG. 7. During the rotation of the tank body 22, the plurality of conical breaking bumps 224 collide and break the animal carcass and the filler, thus accelerating the decomposition process.

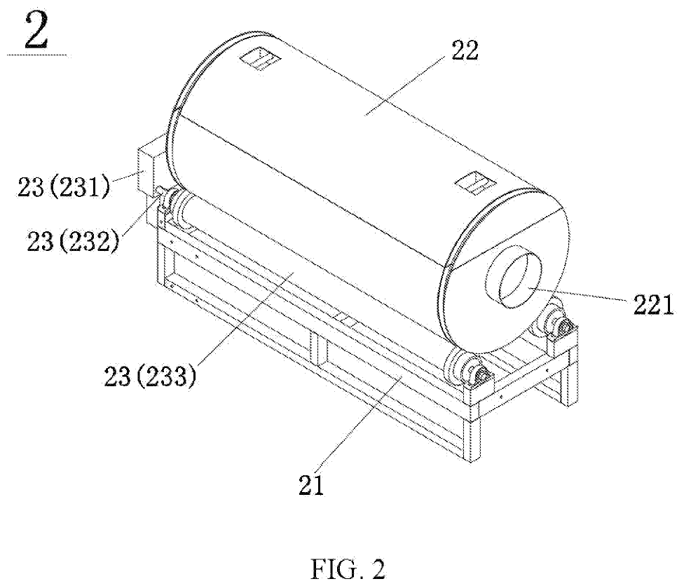

In the present embodiment, as shown in FIG. 2, the rotation driving mechanism includes a motor 231 arranged on the base of the tank body 22 and electrically connected to the central controller, a transmission shaft 232 connected to an output shaft of the motor 231, and at least one transmission roller 233 sleeved on the periphery of the transmission shaft 232 and in contact with the outer wall of the tank body 22. The start and stop of the motor 231 is controlled by the central controller. The motor 231 drives the transmission shaft 232 to rotate, the transmission roller 233 is driven to rotate under the rotation of the transmission shaft 232, and the rotation of the transmission roller 233 makes the tank body 22 rotate.

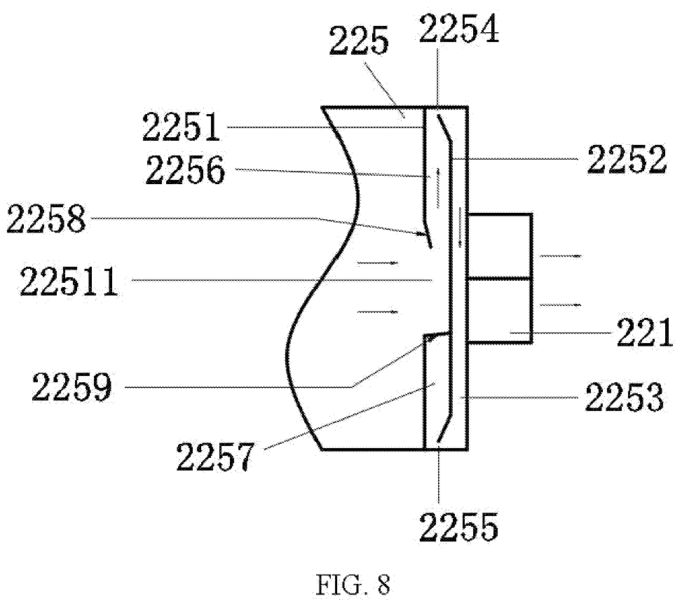

In the present embodiment, as shown in FIG. 8, an exhaust structure 225 is arranged at one end of the tank body 22 near the exhaust port 221. Specifically, the exhaust structure 225 includes a baffle plate 2251 arranged in the tank body 22 and a division plate 2252 arranged in the tank body 22 between the baffle plate 2251 and the exhaust port 221, and an exhaust chamber 2253 is formed between the baffle plate 2251 and the exhaust port 221. An internal exhaust port 22511 is arranged on the baffle plate 2251, a upper exhaust through hole 2254 and a lower exhaust through hole 2255 are formed between the division plate 2252 and the inner wall of the tank body 22. An upper exhaust channel 2256 and a lower exhaust channel 2257 are separately formed between the baffle plate 2251 and the division plate 2252, wherein the upper exhaust channel 2256 is communicated between the internal exhaust port 22511 and the upper exhaust through hole 2254, and the lower exhaust channel 2257 is communicated between the internal exhaust port 22511 and the lower exhaust through hole 2255. At the same time, the internal exhaust port 22511 is respectively provided with a upper movable baffle 2258 swinging in the upper exhaust channel 2256 and a lower movable baffle 2259 swinging in the lower exhaust channel 2257.

In the present embodiment, the upper movable baffle 2258 and the lower movable baffle 2259 are movable plates which can swing freely, that is, when the tank body 22 rotates until the upper movable baffle 2258 is located above the lower movable baffle 2259, the downward swing of the upper movable baffle 2258 makes the internal exhaust port 22511 and the upper exhaust channel 2256 connect to each other, that is, the upper movable baffle 2258 is in the open state, and the waste gas can pass through. While the lower movable baffle 2259 is just against the division baffle 2252, in other words, the lower movable baffle 2259 is in a closed state, so that the internal exhaust port 22511 and the lower exhaust channel 2257 cannot be connected, and the waste gas cannot pass through. When the tank body 22 continues to rotate until the upper movable baffle 2258 is under the lower movable baffle 2259, similarly, the lower movable baffle 2259 swings down to connect the internal exhaust port 22511 and the lower exhaust channel 2257, that is, the lower movable baffle 2259 is in the open state, and the waste gas can pass through. While the upper movable baffle 2258 is just butted with the division plate 2252, that is, the upper movable baffle 2258 is in the closed state, so that the internal exhaust port 22511 is cannot be connected with the upper movable baffle 2258, so that the waste gas cannot pass through. Therefore, the waste gas is always discharged from the upper exhaust channel inside the tank body, while the lower exhaust channel is blocked to prevent the internal substances from entering or even blocking the lower exhaust channel.

After the waste gas produced during fermentation process enters the upper exhaust channel, it enters the exhaust chamber 2253 through the upper exhaust through hole, and is finally discharged from the exhaust port 221 to the waste gas treatment device 3.

In the present embodiment, as shown in FIG. 1, the waste gas treatment device 3 includes an waste gas treatment tank 31, a plurality of waste gas ventilation pipes 32 connected between the front end of the waste gas treatment tank 31 and the plurality of fermentation tanks 2, and a negative pressure exhaust fan 33 arranged at the rear end of the waste gas treatment tank 31. Wherein, an active bacteria bed is arranged at the front side of the interior of the waste gas treatment tank 31, the back side of the interior of the waste gas treatment tank 31 is filled with activated carbon. Specifically, the active bacteria bed is mainly composed of several microbial beds stacked up and down, and the biomembranes are arranged in the microbial beds. Specifically, the biomembranes include plastic carrier, sawdust and microbial active bacteria filled in the plastic carrier.

The waste gas entering the waste gas treatment device 3 enters the waste gas treatment tank 31 through the waste gas ventilation pipe 32, and then the biomembrane in the active bacteria bed in the waste gas treatment tank 31 adsorbs the toxic ingredients and odors in the waste gas to complete the primary treatment of the waste gas. Then, the waste gas after the first treatment passes through the activated carbon, and the pollutants in the primary treated waste gas are adsorbed on the surface of the activated carbon to separate the pollutants from the primary treated waste gas to obtain a colorless, odorless and clean gas. Finally, the treated waste gas is lead to the environment by a negative pressure exhaust fan 33, such that the waste gas treatment is complete and harmless to the environment and human body.

In order to prevent the steam condensation phenomenon in the waste gas ventilation pipe 32, an electric heating component 321 is arranged in the waste gas ventilation pipe 32 in this embodiment. Specifically, the electric heating component 321 can be an electric heating wire. The waste gas ventilation pipe 32 is electrified and heated by the electric heating component 321, so as to properly heat the waste gas ventilation pipe 32, improve the internal temperature, so that the occurrence of steam condensation can be effectively prevented.

In order to achieve the purpose of energy saving, this embodiment makes reasonable use of solar heat. The centralized treatment warehouse 1 is provided with a solar photovoltaic panel 4 which supplies power for the heating component 24, the electric heating component 321 and the negative pressure exhaust fan 33, respectively. The solar photovoltaic panel 4 absorbs sunlight heat, converts it into electrical energy, and supplies power for the entire equipment system.

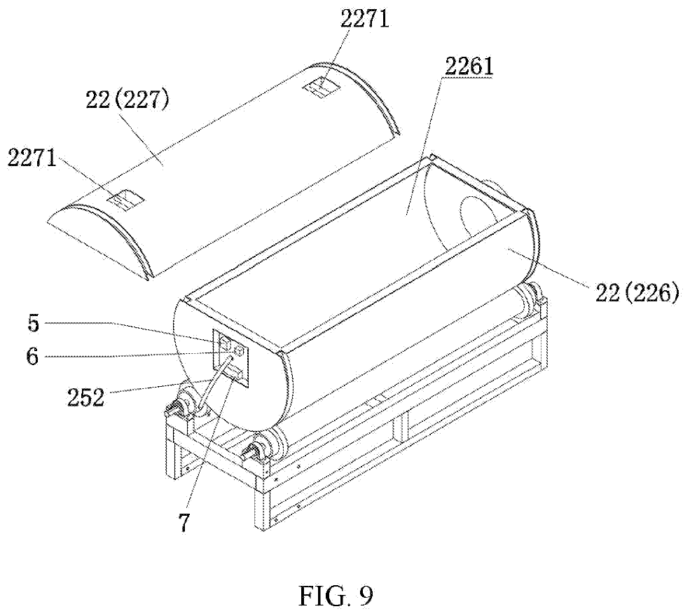

In order to facilitate to put the animal carcasses, filler and fermentation bacteria into the tank body 22, as shown in FIG. 9, the tank body 22 is mainly composed of a lower tank body 226 and a upper cover covered 227 on a lower tank body 227 in the present embodiment. A upper end of the lower tank body is provided with an open input port 2261, and the upper cover 227 is covered on the open input port 2261. The animal carcasses, filler and fermentation bacteria are put into the tank body from the open input port 2261. Meanwhile, the upper cover 227 is provided with a plurality of hooks 2271, which is convenient for automatic lifting of the whole fermentation tank 2.

In order to ensure the tightness of the tank body 22 after the upper cover 227 is covered up the lower tank body 226, a sealing ring is arranged at the edge of the upper end surface of the lower tank body 226 in the present embodiment, so that the upper cover 227 is closely combined with the lower tank body 226, and the upper cover 227 is fixed to the lower tank body 226 by means of screws and other means.

The embodiment of the present disclosure further discloses a fermentation treatment method for various animal carcasses based on the above-mentioned equipment, including the following steps:

(1) Putting a single animal carcass into a numbered fermentation tank 2, adding the fermentation bacteria and filler, wherein the filler can be sawdust. And covering up the fermentation tank 2.
(2) Placing the fermentation tank 2 on a corresponding position of a centralized treatment warehouse 1. Automatic equipment can be used to lift the fermentation tank 2 to the position of centralized processing warehouse 1, and then the fermentation tank 2 can be automatic transferred into the corresponding position or pushed into the corresponding position by robot arm.
(3) Connecting a waste gas ventilation pipe of a waste gas treatment device to an exhaust port of the fermentation tank, and connecting an air pipe of the fermentation tank to an external air supply device.
(4) Introducing air into into a tank body through the air pipe under a control of a central controller, and controlling the heating components inside the tank body to heat the internal temperature of the tank body to 60° C.-70° C. At the same time, the central controller controls a rotation driving mechanism to drive the tank body to rotate at a slow speed for 10-30 days at a rotation rate of 0.083-0.125 r/h. During the rotation of the tank body, the fermentation bacteria in the tank body completely decompose the animal carcass to convert into biological energy, so as to form organic fertilizer and waste gas, and the waste gas is discharged from the exhaust port to the waste gas treatment device.
(5) Purifying waste gas by the waste gas treatment device, and finally discharging the purified waste gas into an external environment.

The step (4) further includes the following steps: detecting, by a temperature sensor, the temperature inside the tank body in real time, and transmitting a detection result to the central controller in real time. When the temperature inside the tank body is detected to exceed 70° C., the central controller disconnects the power supply for the heating component, and the heating component stops working. When the temperature inside the tank body is detected to lower than 60° C., the central controller supplies power to the heating component, and the heating component start to work. Repeating the above step continuously to keep the temperature inside the tank body at 60° C.-70° C.

Of course, the embodiment can also set the corresponding thermometer 5, hygrometer 6 and time recorder 7 on the front end of the tank body 22 of the fermentation tank 2, and the thermometer 5 and the hygrometer 6 are inserted into the tank body 22, as shown in FIG. 9, so that the staff can watch the internal temperature, humidity and fermentation time of the tank body 22 in real time.

The fermentation treatment equipment for various animal carcasses provided by the embodiment can realize the automatic control and management of multiple fermentation tanks with numbers or labels in combination with the stereoscopic warehouse technology, so as to realize the following advantages:

(1) It is automatic operation, so as to improve efficiency and realize automatic storage and retrieval of fermentation tanks;
(2) It has accurate warehouse data management, such that the problem of inaccurate data can be solved, so as to reduce errors, providing more accurate warehouse management;
(3) It provides an accurate inquiry of the real time dynamic inventory information, data processing saves manpower;
(4) It can save storage space, so as to realize the rationalization of high-level warehouse, automatic storage and retrieval and easy operation.
(5) It can save energy and reduce carbon emissions.

The above is only a preferred embodiment of the present disclosure and does not limit the technical scope of the present disclosure. Therefore, other structures obtained by adopting the same or similar technical features as the above-mentioned embodiments of the present disclosure are within the scope of the present disclosure.

What is claimed is:

1. A fermentation treatment equipment for various animal carcasses, comprising a central controller, a centralized processing warehouse, a plurality of fermentation tanks set in the centralized processing warehouse, and at least one waste gas treatment device connected to the plurality of fermentation tanks, wherein each of the plurality of fermentation tanks comprises a tank body base, a tank body arranged on the tank body base, and a rotation driving mechanism which drives the tank body to rotate and is electrically connected to the central controller; an inner wall of the tank body is distributed with a plurality of heating components and a plurality of air supply components controlled by the central controller; a temperature sensor electrically connected to the central controller is provided in the tank body, and a rear end of the tank body is provided with an exhaust port connected to the waste gas treatment device.

2. The fermentation treatment equipment for various animal carcasses according to claim 1, wherein the heating component comprises a heating structure distributed on the inner wall of the tank body along a length direction of the tank body, and at least one heating element distributed in the heating structure along a length direction of the heating structure, wherein the heating element is connected to the the central controller via a power supply wire to realize power supply, or the heating element is powered by a microwave wireless power transmission device communicating with the central controller; the air supply component comprises an air pipe arranged on the inner wall of the tank body and distributed side by side with the heating structure, and a plurality of air holes are formed on side wall of the air pipe; a front end of the tank body is provided with a ventilation pipe connected between the air pipe and an external air supply device, and the ventilation pipe is provided with an electric control valve electrically connected to the central controller.

3. The fermentation treatment equipment for various animal carcasses according to claim 2, wherein a plurality of strip grooves for embedding the heating component and the air supply component are formed on the inner wall of the tank body, and an insulating layer is provided on the inner wall of the tank body.

4. The fermentation treatment equipment for various animal carcasses according to claim 1, wherein the heating component comprises a printed copper foil attached to the inner wall of the tank body, the printed copper foil is connected to the central controller via a power supply wire to realize power supply, or the printed copper foil is powered by a microwave wireless power transmission device communicating with the central controller.

5. The fermentation treatment equipment for various animal carcasses according to claim 1, wherein a plurality of conical breaking bumps are protruded arranged on the inner wall of the tank body.

6. The fermentation treatment equipment for various animal carcasses according to claim 1, wherein the rotation driving mechanism comprises a motor arranged on the tank body base and electrically connected to the central controller, a transmission shaft connected to an output shaft of the motor, and at least one transmission roller sleeved on a periphery of the transmission shaft and in contact with an outer wall of the tank body.

7. The fermentation treatment equipment for various animal carcasses according to claim 1, wherein an exhaust structure is arranged at one end of the tank body near the exhaust port, the exhaust structure comprises a baffle plate arranged in the tank body and a division plate arranged in the tank body between the baffle plate and the exhaust port, and an exhaust chamber is formed between the division plate and the exhaust port, an internal exhaust port is arranged on the baffle plate, an upper exhaust through hole and a lower exhaust through hole are formed between the division plate and the inner wall of the tank body; an upper exhaust channel and a lower exhaust channel are separately formed between the baffle plate and the division plate, wherein the upper exhaust channel is communicated between the internal exhaust port and the upper exhaust through hole, and the lower exhaust channel is communicated between the internal exhaust port and the lower exhaust through hole; and the internal exhaust port is respectively provided with a upper movable baffle swinging in the upper exhaust channel and a lower movable baffle swinging in the lower exhaust channel.

8. The fermentation treatment equipment for various animal carcasses according to claim 1, wherein the tank body is mainly composed of a lower tank body and an upper cover covered on a lower tank body; an upper end of the lower tank body is provided with an open input port, and the upper cover is covered on the open input port; and the upper cover is provided with a plurality of hooks.

9. The fermentation treatment equipment for various animal carcasses according to claim 1, wherein the waste gas treatment device comprises an waste gas treatment tank, a plurality of waste gas ventilation pipes connected between a front end of the waste gas treatment tank and the plurality of fermentation tanks, and a negative pressure exhaust fan arranged at a rear end of the waste gas treatment tank, wherein an active bacteria bed is arranged at a front side of an interior of the waste gas treatment tank, a back side of the interior of the waste gas treatment tank is filled with activated carbon; an electric heating component is provided inside the waste gas ventilation pipe; the centralized treatment warehouse is provided with a solar photovoltaic panel which supplies power for the heating component, the electric heating component and the negative pressure exhaust fan, respectively.

10. A fermentation treatment method for animal carcasses, comprising:
(1) putting a single animal carcass into a numbered fermentation tank, adding a fermentation bacteria and filler, and covering up the fermentation tank;
(2) placing the fermentation tank on a shelf to a corresponding position of a centralized treatment warehouse;
(3) connecting a waste gas ventilation pipe of a waste gas treatment device to an exhaust port of the fermentation tank, and connecting an air pipe of the fermentation tank to an external air supply device;
(4) introducing air into into a tank body through the air pipe under a control of a central controller, and controlling internal heating components to heat a internal temperature of the tank body to 60° C.-70° C.; wherein the central controller controls a rotation driving mechanism to drive the tank body to rotate at a slow speed for 10-30 days; during a rotation of the tank body, the fermentation bacteria in the tank body completely decompose and decay the animal carcass to convert into biological energy, so as to form organic fertilizer and waste gas, and the waste gas is discharged from the exhaust port to the waste gas treatment device;
(5) purifying waste gas by the waste gas treatment device, and finally discharging the purified waste gas into an external environment.

11. The fermentation treatment method for animal carcass according to claim 10, wherein the step (4) further comprises following steps: detecting, by a temperature sensor, the temperature inside the tank body in real time, and transmitting a detection result to the central controller in real time; disconnecting power supply for the heating component by the central controller when the temperature inside the tank body is detected to exceed 70° C., and the heating component stops working; supplying power to the heating component by the central controller when the temperature inside the tank body is detected to lower than 60° C., and the heating component start to work; repeating this step continuously to keep the temperature inside the tank body at 60° C.-70° C.
